(19) United States
(12) Patent Application Publication
(10) Pub. No.: US 2021/0204812 A1
(43) Pub. Date: Jul. 8, 2021

(54) BIOSENSOR

(71) Applicant: Yamaha Corporation, Hamamatsu (JP)

(72) Inventor: Yuki UEYA, Hamamatsu (JP)

(21) Appl. No.: 17/189,037

(22) Filed: Mar. 1, 2021

Related U.S. Application Data
(63) Continuation of application No. PCT/JP2019/030942, filed on Aug. 6, 2019.

(30) Foreign Application Priority Data
Sep. 3, 2018 (JP) 2018-164918

(51) Int. Cl. A61B 5/00; H01L 41/047; H01L 41/053; H01L 41/113; H01L 41/193

(52) U.S. Cl. CPC A61B 5/00; H01L 41/0475; H01L 41/0533; A61B 5/02108; H01L 41/193; A61B 2560/0443; A61B 2562/18; H01L 41/1132

(57) ABSTRACT

A biosensor includes a sheet-like piezoelectric element, a spacer disposed around the piezoelectric element in plan view with a gap therebetween, and a covering member that covers a front side of each of the spacer and the piezoelectric element. The spacer supports the covering member from a rear side of the covering member, and the piezoelectric element is fixed to the covering member.

### CROSS-REFERENCE TO RELATED APPLICATIONS

This application is a continuation application of International Application No. PCT/JP2019/030942, filed on Aug. 6, 2019, which claims priority to Japanese Patent Application No. 2018-164918 filed in Japan on Sep. 3, 2018. The entire disclosures of International Application No. PCT/JP2019/030942 and Japanese Patent Application No. 2018-164918 are hereby incorporated herein by reference.

### BACKGROUND

### Technological Field

The present invention relates to a biosensor.

### Background Information

Vibrations generated inside the living body, such as, e.g., the heartbeat, pulse waves, blood flow sounds, and respiratory sounds (not limited to sonic vibrations in the audible range, and including low frequency vibrations and ultrasonic vibrations in the non-audible range) can be measured or observed in order to carry out diagnoses, health management, etc.

An example of a known biosensor that detects vibrations in the living body is a vibration waveform sensor using piezoelectric elements (refer to International Publication No. 2017/187710). This known vibration waveform sensor has a piezoelectric element mounted on a substrate, a spacer disposed around the piezoelectric element, and a cover portion covering the piezoelectric element; the area surrounded by the spacer is filled with a silicon resin, for example. In this conventional biosensor (vibration waveform sensor), the cover portion side is brought into contact with the living body in order to detect vibrations in the living body.

However, in the conventional biosensor, since vibrations transmitted to the piezoelectric element from the spacer via the substrate are primarily detected, the propagation path is long. As a result, the sensitivity tends to decrease and the signal tends to become mixed with noise. Moreover, due to the elasticity of the cover portion and the silicon resin that are present between the living body to be measured and the piezoelectric sensor, the vibrations tend to become attenuated. Also, since the spacer and the cover portion surround and are in close contact with the piezoelectric element, deformations of the piezoelectric element are suppressed. From these standpoints as well, the sensitivity of the conventional biosensor tends to decrease. Therefore, there is a demand for a highly sensitive biosensor with robust noise resistance.

### SUMMARY

In light of the circumstances described above, an object of this disclosure is to provide a highly sensitive biosensor with robust noise resistance.

A biosensor according to one aspect of this disclosure for solving the problem described above comprises a sheet-like piezoelectric element, a spacer disposed around the piezoelectric element in plan view with a gap therebetween, and a covering member that covers a front side of each of the spacer and the piezoelectric element. The spacer supports the covering member from a rear side of the covering member, and the piezoelectric element is fixed to the covering member.

### BRIEF DESCRIPTION OF THE DRAWINGS

FIG. 1 is a schematized bottom view showing the rear surface of a biosensor according to one embodiment.

FIG. 2 is a schematized cross section through line A-A of the biosensor of FIG. 1.

FIG. 3 is a schematized bottom view showing the rear surface of a biosensor according to a different embodiment than that of FIG. 1.

FIG. 4 is a schematized cross section of a biosensor according to a different embodiment than that of FIGS. 1 and 3.

### DETAILED DESCRIPTION OF THE EMBODIMENTS

Selected embodiments will now be explained in detail below, with reference to the drawings as appropriate. It will be apparent to those skilled from this disclosure that the following descriptions of the embodiments are provided for illustration only and not for the purpose of limiting the invention as defined by the appended claims and their equivalents.

The biosensor according to one aspect of this disclosure comprises a sheet-like piezoelectric element, a spacer disposed around the piezoelectric element in plan view with a gap therebetween, and a covering member that covers the front side of the spacer and the piezoelectric element, wherein the spacer supports the covering member from the rear side, and the piezoelectric element is fixed to the covering member.

In the biosensor, the rear side is the side that faces the surface of a living body in which vibrations are to be detected, and the front side is the side opposite to the rear side.

In the biosensor, the rear surface of the spacer is preferably a plane that is parallel to the rear surface of the piezoelectric element.

The biosensor preferably further comprises a plate disposed on the rear side of the piezoelectric element so as to oppose the covering member.

In the biosensor, the rear surface of the plate preferably projects to the rear side from the rear surface of the spacer.

The biosensor preferably comprises a plurality of the piezoelectric elements arranged so as not to overlap each other in plan view.

In the biosensor, the mean (average) thickness of the spacer is preferably 300 μm or more and 800 μm or less.

In this disclosure, “rear side” means the side positioned facing toward the surface of the living body, and the “front side” means the side opposite the “rear side,” that is, the side positioned opposite the surface of the living body and facing away from the surface of the living body. “Mean thickness” means the thickness averaged over measurements at ten arbitrary points. “Plan view” is as viewed from the front side of the living body to the rear side of the living body.

In the biosensor, the covering member to which the piezoelectric element is fixed is supported by the spacer. Thus, in the biosensor, because the piezoelectric element can be brought into contact with the living body to detect vibrations of the living body, the propagation path can be shortened. Moreover, in the biosensor, there is a gap between the piezoelectric element and the spacer. For this reason, since the deformation of the piezoelectric element tends not to be suppressed by the spacer, or the like, the sensitivity of the piezoelectric element can be easily secured. Accordingly, the biosensor is highly sensitive and has robust noise resistance.

An embodiment of this disclosure will be described in detail below with reference to the drawings as deemed appropriate.

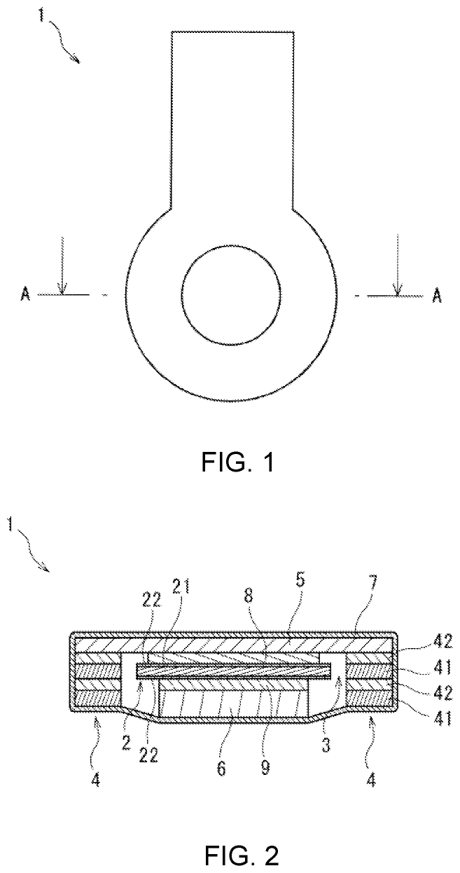

FIGS. 1 and 2 show a biosensor 1 according to one embodiment of this disclosure. The biosensor 1 is disposed in close contact with the surface of a living body, such as that of an animal, and is used for detecting vibrations inside the living body, such as a pulse wave.

The biosensor 1 comprises a sheet-like piezoelectric element 2, a spacer 4 disposed around the piezoelectric element 2 in plan view with a gap 3 therebetween, a covering member 5 that covers the front side of the spacer 4 and the piezoelectric element 2, a plate 6 disposed on the rear side of the piezoelectric element 2 so as to oppose the covering member 5, and a shield layer 7 disposed on the outermost side enclosing all components.

### Piezoelectric Element

The piezoelectric element 2 is formed from a piezoelectric material that converts pressure into voltage, and converts the deformation caused by the force applied by a pressure wave of biological vibration into voltage. The piezoelectric element 2 has a sheet-like or film-like piezoelectric body 21 and a pair of electrodes 22 overlaid on the front and back of the piezoelectric body 21.

### Piezoelectric Body

The piezoelectric material forming the piezoelectric body 21 can be an inorganic material, such as lead zirconate titanate, but is preferably a polymer piezoelectric material having flexibility so as to be capable of coming into close contact with the surface of a living body. Further, by using a porous film, wherein a large number of pores are formed in the polymer piezoelectric material as the piezoelectric body 21, the flexibility and the piezoelectric constant can be made relatively high.

Examples of the polymer piezoelectric material include polyvinylidene fluoride (PVDF), poly(vinylidene fluoride-trifluoroethylene), and poly(vinylidenecyanide-vinylacetate) (P(VDCN))/VAc. Further, by forming these polymer piezoelectric materials into a porous film, the piezoelectric element 2 can be produced with greater flexibility and a higher piezoelectric constant.

Further, a piezoelectric body obtained by forming a large number of flat pores in polyethylene terephthalate (PTFE), polypropylene (PP), polyethylene (PE), polyethylene terephthalate (PET), or the like, which do not have piezoelectric properties, and applying piezoelectric properties by polarizing and charging the opposing flat porous surfaces by means of corona discharge, or the like, can be used as the piezoelectric body 21.

The lower limit of the mean thickness of the piezoelectric body 21 is preferably 10 nm, and more preferably 50 nm. The upper limit of the mean thickness of the piezoelectric body 21, on the other hand, is preferably 500 nm, and more preferably 200 nm. If the mean thickness of the piezoelectric body 21 is less than the aforementioned lower limit, there is the risk that the strength of the piezoelectric element 2 will be insufficient. Conversely, if the mean thickness of the piezoelectric body 21 exceeds the aforementioned upper limit, the deformability of the piezoelectric element 2 decreases, so that the detection sensitivity may become insufficient.

### Electrode

Electrodes 22 are overlaid on both sides of the piezoelectric body 21 and are used to detect the potential difference between the front and back of the piezoelectric body 21.

Any material exhibiting conductivity can be used for the electrode 22, such as metals such as aluminum, copper, and nickel, and carbon and the like.

The mean thickness of the electrode 22 is not particularly limited, and can be at least 0.1 μm and up to 30 μm, depending on the overlaying method. If the mean thickness of the electrode 22 is less than the aforementioned lower limit, there is the risk that the strength of electrode 22 will be insufficient. Conversely, if the mean thickness of the electrode 22 exceeds the aforementioned upper limit, there is the risk that the transmission of vibrations to the piezoelectric body 21 will be hindered.

The method of overlaying the electrode 22 onto the piezoelectric body 21 is not particularly limited, and examples include metal deposition, printing carbon conductive ink, and the coating and drying of silver paste.

The electrode 22 can be formed divided into a plurality of areas in plan view to cause the piezoelectric element 2 to effectively function as a plurality of piezoelectric elements.

Although the electrode 22 is formed up to the outer edge of the piezoelectric element 2 in FIG. 2, the area where the electrode 22 is formed need not reach the outer edge of the piezoelectric element 2. That is, the electrodes 22 can be overlaid on the entire front and rear surfaces of the piezoelectric body 21, or can be overlaid on a portion of the front or rear surface of the piezoelectric body 21, as long as the potential difference can be detected.

The plan-view shape of the piezoelectric element 2 can be a circle with a diameter of 2 mm or more and 10 mm or less. If the diameter is less than the aforementioned lower limit, it may become difficult to position the biosensor 1 such that the piezoelectric element 2 covers a blood vessel when pulse waves are measured, for example. Conversely, if the diameter exceeds the aforementioned upper limit, the biosensor 1 will become unduly large, making handling inconvenient.

Signal wiring 8 is disposed on the front surface of the piezoelectric element 2, that is, between the covering member 5 and the electrode 22 on the front side of the piezoelectric element 2. In addition, ground wiring 9 is disposed on the rear surface of the piezoelectric element 2, that is, between the plate 6 and the electrode 22 on the rear side of the piezoelectric element 2.

This signal wiring 8 and ground wiring 9 are used for transmitting the potential difference detected by the pair of electrodes 22 of the piezoelectric element 2 to a detection circuit. Therefore, the signal wiring 8 and the ground wiring 9 are connected to the detection circuit, which is not shown.

The signal wiring 8 and the ground wiring 9 can be made of any conductive material, such as a film made of a metal such as aluminum, copper, or nickel, a film containing a conductive material such as carbon, or a woven or nonwoven fabric made of conductive fibers.

The mean thicknesses of the signal wiring 8 and the ground wiring 9 are not particularly limited and can be 15 μm or more and 50 μm or less. If the mean thicknesses of the signal wiring 8 and the ground wiring 9 are less than the aforementioned lower limit, the conductivity of the signal wiring 8 and the ground wiring 9 may become insufficient. Conversely, if the mean thicknesses of the signal wiring 8 and the ground wiring 9 exceed the aforementioned upper limit, there is the risk that transmission of vibrations to the piezoelectric element 2 will be hindered.

In the biosensor 1, the piezoelectric element 2 is fixed to the covering member 5 described further below. That is, an elastic member such as a spring or rubber which biases the piezoelectric element 2 to the front side or rear side, for example, is not placed between the piezoelectric element 2 and the covering member 5. By fixing the piezoelectric element 2 to the covering member 5 in this manner, the vibrations from the living body can be prevented from being absorbed by the elastic member, so that the sensitivity of the piezoelectric element 2 can be increased. As shown in FIG. 2, the piezoelectric element 2 is fixed to the covering member 5 with the signal wiring 8 sandwiched therebetween. In this way, the piezoelectric element 2 and the covering member 5 can also be fixed with a fixing member that does not have elasticity like, for example, a spring or rubber, interposed therebetween. Examples of the fixing member sandwiched between the piezoelectric element 2 and the covering member 5 include conductive wiring using a conductive film or the like, or a member for adjusting the thickness of the biosensor.

### Spacer

The spacer 4 is configured by overlaying wall 41 and the ground wiring 42, as shown in FIG. 2, for example. The spacer 4 is not limited to the configuration of FIG. 2, and can be composed of only the wall 41, for example, but the configuration of FIG. 2 will be used in the description below.

Examples of the material of the wall 41 of the spacer 4 include polyethylene terephthalate (PET), polypropylene (PP), polyethylene (PE), polyethylene naphthalate (PEN), polyarylate (PAR), polyimide (PI), and the like, of which PET having an appropriate rigidity is preferred.

The material of the ground wiring 42 can be the same as that of the ground wiring 9 of the piezoelectric element 2. In addition, the ground wiring 42 should be disposed such that its height position (the position in the front-back direction of the biosensor 1) matches those of the signal wiring 8 disposed on the front surface of the piezoelectric element 2 and the ground wiring 9 disposed on the rear surface of the piezoelectric element 2. Specifically, the ground wiring 42 of the spacer 4 should have the same thickness as that of the signal wiring 8 and the ground wiring 9 at the corresponding height, and the wall 41 sandwiched by the ground wiring 42 should have the same thickness as the piezoelectric element 2. By means of such an arrangement, the ground wiring 42 of the spacer 4 functions as a shield in order to prevent noise from being mixed into the signal detected by the piezoelectric element 2. In addition, when the biosensor 1 is fabricated, the ground wiring 9 and the signal wiring 8 of the piezoelectric element 2, and the ground wiring 42 of the spacer 4 can be overlaid at once in the same layer, so that the manufacturing efficiency can be improved.

The spacer 4 supports the covering member 5, described further below, from the rear side. That is, since the covering member 5 is fixed in position by the spacer 4, the covering member is prevented from vibrating. As a result, it is possible to increase the sensitivity of the piezoelectric element 2 fixed to the covering member 5.

The spacer 4 can be arranged at intervals around the piezoelectric element 2 as long as it is possible to support the covering member 5, but is preferably arranged so as to surround the entire circumference of the piezoelectric element 2 in plan view. By arranging the spacer 4 so as to surround the entire circumference of the piezoelectric element 2 in plan view in this manner, the covering member 5 can be stably supported and the sensitivity of the piezoelectric element 2 can be further increased.

In addition, the rear surface of the spacer 4 is preferably a plane that is parallel to the rear surface of the piezoelectric element 2. By configuring the rear surface of the spacer 4 to be parallel to the rear surface of the piezoelectric element 2 in this manner, the contact area of the spacer 4 with respect to a living body becomes large when the biosensor 1 is brought in contact with a living body, so that the covering member 5 can be stably supported. Thus, it is possible to further increase the sensitivity of the piezoelectric element 2.

The thickness of the spacer 4 is set to a thickness such that the rear surface of the spacer 4 can come into contact with a living body and fix the covering member 5 when the biosensor 1 is used. In addition, the thickness of the spacer 4 is adjusted such that the piezoelectric element 2 can detect vibrations from the rear side when the biosensor 1 is used, that is, such that the piezoelectric element 2, the plate 6, the shield layer 7, and the living body are continuous in a direction from the front side to the rear side (hereinafter also referred to as “rearward direction”) regardless of the state of the biological vibrations. “Continuous in the rearward direction regardless of the state of the biological vibrations” means that, for example, even if the piezoelectric element 2 receives a compression force due to biological vibrations, a gap is not formed between the plate 6 and the piezoelectric element 2, for example.

The lower limit of the mean thickness of the spacer 4 is preferably 300 μm, and more preferably 400 μm. The upper limit of the mean thickness of the spacer 4, on the other hand, is preferably 800 μm, and more preferably 700 μm. If the mean thickness of the spacer 4 is less than the aforementioned lower limit, when the biosensor 1 is brought into contact with a living body, the plate 6 can protrude excessively from the rear surface of the spacer 4 so that the spacer 4 cannot contact the living body and the covering member 5 cannot be supported. Conversely, if the mean thickness of the spacer 4 exceeds the aforementioned upper limit, for example, shaking on the rear side of the spacer 4 is amplified on the front side, with the thickness of the spacer 4 as the radius. Accordingly, there is the risk that the covering member 5 tends to vibrate.

The mean width of the rear surface of the spacer 4 (mean width in the radial direction) is not particularly limited, but can be set to 1 mm or more and 5 mm or less, for example. If the mean width of the spacer 4 is less than the aforementioned lower limit, when the biosensor 1 is brought into contact with a living body, the contact area of the spacer 4 becomes small, so that it may not be possible to stably support the covering member 5. Conversely, if the mean width of the spacer 4 exceeds the aforementioned upper limit, the biosensor 1 becomes unduly large in plan view, and handling may become inconvenient.

There is a gap 3 between the spacer 4 and the piezoelectric element 2. The gap 3 need only be of such size that no contact is made with the spacer 4 even when the piezoelectric element 2 deforms, and the lower limit of the width of the gap 3 can be set to 10 μm, for example. The upper limit of the width of the gap 3, on the other hand, although not particularly limited, can be set to 3 mm, for example, from the standpoint of handling capability of the biosensor 1, that is, miniaturization.

The gap 3 is not filled with a filler, such as gel. Not filling the gap 3 with a filler makes it possible to avoid the suppression of deformations of the piezoelectric element 2, such that the sensitivity of the piezoelectric element is easily secured.

### Covering Member

The covering member (cover) 5 is plate-shaped and covers the front side of the spacer 4 and the piezoelectric element 2, as described above. The covering member 5 can cover the front side of the spacer 4 and the piezoelectric element 2 so as to surround the outer edge of the spacer 4 in plan view; preferably, however, coverage is such that the outer edge of the covering member 5 and the outer edge spacer 4 coincide. This type of coverage makes it possible to reduce the size of the covering member 5, so that the handling capability of the biosensor 1 is improved.

The material of the covering member 5 can be the same as that of the wall 41 of the spacer 4. Further, the covering member 5 preferably exhibits flexibility. Providing the covering member 5 with a certain degree of flexibility in this way makes it possible to cause the biosensor 1 to be appropriately contacted, even if the surface of the living body to be measured is a curved surface.

The lower limit of the mean thickness of the covering member 5 is preferably 50 μm, and more preferably 100 μm. The upper limit of the mean thickness of the covering member 5, on the other hand, is preferably 400 μm, and more preferably 250 μm. If the mean thickness of the covering member 5 is less than the aforementioned lower limit, the covering member 5 tends to bend too much, so that it becomes difficult to fix the position of the piezoelectric element 2. For this reason, the sensitivity of the biosensor 1 could be reduced. Further, if the mean thickness of the covering member 5 is less than the aforementioned lower limit, the parasitic capacitance may increase, and there may be a risk that noise tends to be generated. Conversely, if the mean thickness of the covering member 5 exceeds the aforementioned upper limit, the flexibility of the covering member 5 would be insufficient, and if the surface of the living body to be measured is a curved surface, it may be difficult to bring the biosensor 1 into appropriate contact.

### Plate

The plate 6 transmits the vibrations which are generated in part of a living body and propagated from the living body to the piezoelectric element 2 as a vibration of the entire surface of the plate 6. It is possible to increase the sensitivity of the piezoelectric element 2 by transmitting the vibration to the piezoelectric element 2 as a wide-area vibration in this manner.

In the biosensor 1, the plate 6 is smaller than the piezoelectric element 2 in plan view. That is, the piezoelectric element 2 projects to the outside of the plate 6 in plan view. On the other hand, the plate 6 can be made larger than the piezoelectric element 2 in plan view. That is, the plate 6 can be configured to project to the outside of the piezoelectric element 2 in plan view.

In addition, in the case that the plate 6 is smaller than the piezoelectric element 2 in plan view, the plate 6 can be smaller than the electrode 22 of the piezoelectric element 2 in plan view, and can come in contact with the piezoelectric element 2 in an area that is narrower than the electrode 22. On the other hand, the plate 6 can be made larger than the electrode 22 of the piezoelectric element 2 in plan view, that is, come in contact with the piezoelectric element 2 in an area that is wider than the electrode 22.

Preferably, the rear surface of the plate 6 is flush with the rear surface of the spacer 4, or the rear surface of the plate 6 projects rearward from the rear surface of the spacer 4. Configuring the plate 6 in this manner makes it possible for the piezoelectric element 2 to receive more reliably the vibrations from a living body in a state in which the rear surface of the spacer 4 is in contact with the living body.

The material of the plate 6 can be the same as that of the wall 41 of the spacer 4. The plan-view shape of the plate 6 is preferably the same as the plan-view shape of the piezoelectric element 2. The mean thickness of the plate 6 can be the same as that of the covering member 5.

### Shield Layer

The shield layer 7 is disposed on the outermost side of the biosensor 1 so as to enclose all components, as described above. That is, the shield layer 7 is disposed so as to surround the piezoelectric element 2, the spacer 4, the covering member 5, and the plate 6.

The shield layer 7 has an insulating layer and a conductive layer that is overlaid to the outer surface of the insulating layer. An acrylic can be used as the insulating layer, for example. The conductive layer can be a coating layer of a conductive coating material such as silver or copper. Application of an insulating layer to the inner surface of the shield layer 7 and a conductive layer to the outer surface makes it possible to suppress short-circuiting of the piezoelectric element 2, and to provide a shield against noise.

In addition, the shield layer 7 is preferably flexible. Since the shield layer 7 exhibits flexibility, the vibrations generated in a living body can be more reliably transmitted to the plate 6.

Although not particularly limited, the mean thickness of the shield layer 7 can be, for example, 10 μm or more and 100 μm or less. If the mean thickness of the shield layer 7 is less than the aforementioned lower limit, the shield layer 7 could tend to tear during use. Conversely, if the mean thickness of the shield layer 7 exceeds the aforementioned upper limit, the flexibility of the shield layer 7 may be insufficient, and the sensitivity of the biosensor 1 may be reduced.

### Biosensor Manufacturing Method

The biosensor 1 can be manufactured by means of a manufacturing method including, for example, a signal wiring overlaying step, a piezoelectric element overlaying step, a ground wiring overlaying step, a plate overlaying step, and a shield layer coating step. Signal wiring overlaying step

In the signal wiring overlaying step, the signal wiring 8 is overlaid to the rear surface of the covering member 5. Specifically, a thin metal film in the form of the signal wiring 8 is attached to the rear surface of the covering member 5 by means of an adhesive. At this time, the ground wiring 42 on the front side of the spacer 4 is simultaneously overlaid. Piezoelectric element overlaying step

In the piezoelectric element overlaying step, the piezoelectric element 2 is overlaid to the rear surface of the signal wiring 8 overlaid in the signal wiring overlaying step. Specifically, the piezoelectric element 2 is attached to the rear surface of the signal wiring 8 by means of an adhesive. At this time, the wall 41 of the spacer 4, which is at the same height position as the piezoelectric element 2, is simultaneously overlaid to the ground wiring 42.

### Ground Wiring Overlaying Step

In the ground wiring overlaying step, the ground wiring 9 is overlaid to the rear surface of the piezoelectric element 2 overlaid in the piezoelectric element overlaying step. Specifically, a thin metal film in the form of the ground wiring 9 is attached to the rear surface of the piezoelectric element 2 by means of an adhesive. At this time, the ground wiring 42 on the rear side of the spacer 4 is simultaneously overlaid on the wall 41. Since the ground wiring 9 overlaid on the rear surface of the piezoelectric element 2 and the ground wiring 42 of the spacer 4 are at the same potential, the two are preferably connected to each other.

### Plate Overlaying Step

In the plate overlaying step, the plate 6 is overlaid on the rear surface of the ground wiring 9 overlaid in the ground wiring overlaying step. Specifically, the plate 6 is attached to the rear surface of the ground wiring 9 by means of an adhesive. At this time, the wall 41 of the spacer 4 at the same height position as the plate 6 is simultaneously overlaid.

### Shield Layer Coating Step

In the shield layer coating step, the shield layer 7 is coated so as to surround the piezoelectric element 2, the spacer 4, the covering member 5, and the plate 6, after the plate overlaying step.

The biosensor 1 can be manufactured by means of the foregoing steps. In the manufacturing method described above, a method was described in which the covering member 5 and the signal wiring 8 are bonded, and the ground wiring 9 and the plate 6 are bonded, but the configuration can be such that the foregoing are not bonded, and the signal wiring 8, the piezoelectric element 2, and the ground wiring 9 are sandwiched between the covering member 5 and the plate 6. By means of such a configuration, deformation of the piezoelectric element 2 tends not to be suppressed in comparison with a case in which they are bonded, and the sensitivity of the piezoelectric element 2 is readily secured.

### Method of Using the Biosensor

The biosensor 1 is used by being fixed to a living body such that the rear surface of the spacer 4 comes into contact with the living body.

The fixing position of the biosensor 1 to a living body is a location where biological vibrations are generated and that overlaps the piezoelectric element 2 in plan view. In practice, since the piezoelectric element 2 has a certain size, a method in which the biosensor 1 is disposed at a location where biological vibrations are assumed to occur is used to confirm that biological vibrations can be detected and can be used as a method for positioning the biosensor 1. If biological vibrations cannot be detected at such a location, the placement position can be changed to carry out the confirmation procedure again.

In addition, there can be cases in which a living body is a curved surface at the fixing position to the living body; in such a case, the covering member 5 can be bent along the curved surface of the living body.

The method for fixing the biosensor 1 to a living body is not particularly limited, but can be adhesion by means of tape, or the like. In the biosensor 1, the biosensor 1 can be fixed in a state of being pressed against the living body to the extent that the position of the covering member 5 is fixed by means of the spacer 4. Thus, it is not necessary to fix the biosensor 1 to the living body with a large pressing force.

According to the biosensor 1 fixed as described above, it is possible to observe the displacement of electric potential of the piezoelectric element 2 corresponding to the biological vibration. It is possible to observe the amplitude, period, etc., of the vibration of the living body, by measuring this potential displacement by means of a known measuring device.

### Advantages

In the biosensor 1, the covering member 5 to which the piezoelectric element 2 is fixed is supported by the spacer 4. For this reason, since, in the biosensor 1, the piezoelectric element 2 can be brought into contact with a living body to detect vibrations of the living body, the propagation path can be shortened. In addition, the biosensor 1 has the gap 3 between the piezoelectric element 2 and the spacer 4. For this reason, since the deformation of the piezoelectric element 2 tends not to be suppressed by the spacer 4, or the like, the sensitivity of the piezoelectric element 2 can be easily secured. Therefore, the biosensor 1 has high sensitivity and robust noise resistance.

### Second Embodiment

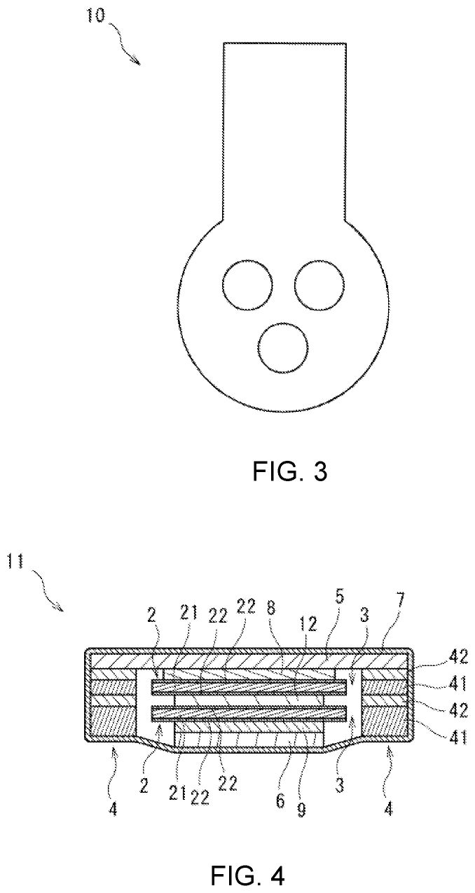

FIG. 3 shows a biosensor 10 according to one embodiment of this disclosure. The biosensor 10 is disposed, for example, in close contact with the surface of a living body, such as that of an animal, and is used for detecting vibrations inside the living body, such as a pulse wave.

The biosensor 10 comprises three sheet-like piezoelectric elements, three spacers, a covering member, three plates, and a shield layer. The three spacers are disposed around the three piezoelectric elements in plan view, respectively, and there exists a gap between each of the three piezoelectric elements and a corresponding spacer. The covering member covers the front sides of the three spacers and the three piezoelectric elements. The three plates are disposed on the rear sides of the three piezoelectric elements, respectively, so as to face toward the covering member. The shield layer is disposed on the outermost side enclosing all components.

### Piezoelectric Element

The shape of each of the piezoelectric elements in plan view can be that of a circle with a diameter of 2 mm or more and 10 mm or less.

The three piezoelectric elements are disposed so as not to overlap in plan view. While the arrangement positions of the three piezoelectric elements are not particularly limited, for example, they are arranged as shown in FIG. 3 such that the centers thereof form an equilateral triangle, the equal sides of which are between 5 mm and 15 mm, inclusive.

In addition, the three piezoelectric elements are preferably connected in parallel. The parallel connection of the three piezoelectric elements makes it possible for the biosensor 10 to detect vibrations as long as any one of the piezoelectric elements detects vibrations of a living body. For this reason, the positioning of the biosensor 10 can be easily carried out.

The piezoelectric elements can be configured in the same manner as the piezoelectric element 2 according to the first embodiment, other than the plan-view shape thereof described above, so that a detailed description will be omitted.

### Spacers and Plates

The spacers and plates can be configured in the same manner as the spacer 4 and the plate 6 according to the first embodiment with respect to each of the three piezoelectric elements, so that a detailed description will be omitted.

### Covering Member

The covering member has the shape of one plate, and covers the front sides of the three piezoelectric elements and the spacers. The covering member can be configured in the same manner as the covering member 5 according to the first embodiment, so that a detailed description will be omitted.

### Shield Layer

The shield layer can be configured in the same manner as the shield layer 7 according to the first embodiment, so that a detailed description will be omitted.

The biosensor 10 can be manufactured and used in the same manner as the biosensor 1 according to the first embodiment. Therefore, a detailed description thereof will be omitted.

### Advantages

Since the biosensor 10 includes a plurality of piezoelectric elements arranged so as not to overlap in plan view, the area of each piezoelectric element in plan view can be reduced compared to the case in which one piezoelectric element is provided. Since the vibrations of a living body are generated in one given location, and the area of the piezoelectric element that comes in contact with the biological vibrations is small, the surface pressure generated in the piezoelectric element as a result of the biological vibrations can be increased. Therefore, the biosensor 10 can be made more sensitive to biological vibrations. In addition, since the area of each piezoelectric element in plan view is small, even if the measurement position of the living body is a curved surface, it is easy to fix the biosensor 10 along the curved surface.

### Other Embodiments

The above-described embodiments do not limit the configuration of this disclosure. Therefore, in the above-described embodiments, the composition of the elements of each part of the embodiment can be omitted, replaced, or added to based upon the recitation of the present Specification and common knowledge of the art, all of which shall be interpreted as belonging to the scope of this disclosure.

In the embodiments described above, a case in which the biosensor has a shield layer was described, but a shield layer is not an essential required component and can be omitted.

In the embodiments described above, a case in which the biosensor has a plate was described, but a plate is not an essential required component and can be omitted. A biosensor that does not have a plate directly detects vibrations by means of the piezoelectric element.

In the embodiments described above, a case was illustrated in which the areas of the wall of the spacer and the ground wiring in plan view are equivalent, but these plan-view areas can be different depending on the position in the height direction.

In the embodiments described above, a case was described in which signal wiring is disposed on the front surface of the piezoelectric element and ground wiring is disposed on the rear surface of the piezoelectric element, but the arrangement of the signal wiring and the ground wiring can be reversed, that is, the signal wiring can be disposed on the rear surface of the piezoelectric element and the ground wiring can be disposed on the front surface of the piezoelectric element.

In the second embodiment described above, the case was described in which there are three piezoelectric elements arranged so as not to overlap in plan view, but the number of the piezoelectric elements arranged so as not to overlap in plan view is not limited to three, and can be two, or four or more, which include one piezoelectric element and at least one additional piezoelectric element that does not overlap the one piezoelectric element in plan view.

In addition, as shown in FIG. 4, a biosensor 11 can comprise a plurality of piezoelectric elements 2 (there are two piezoelectric elements 2 in FIG. 4) that are overlaid on the rear surface of the covering member 5. In the biosensor 11 shown in FIG. 4, two piezoelectric elements 2 are connected in series via connection wiring 12. It is possible to increase the sensitivity of the piezoelectric element 2 by overlaying a plurality of the piezoelectric elements 2 in series in this manner.

In the embodiments described above, the case was described in which the plan-view shape of the piezoelectric element is a circle, but the plan-view shape of the piezoelectric element is not limited to a circle. The plan-view shape of the piezoelectric element can be, for example, an ellipse, or a polygon such a triangle, a quadrilateral, a pentagon, or a hexagon. The plan-view shape of the piezoelectric element is appropriately determined so that the piezoelectric element is efficiently arranged. In addition, if the biosensor has a plurality of piezoelectric elements, the plan-view shapes thereof can all be the same, or, some or all of the shapes can be different.

The biosensor according to this disclosure can be used to measure various vibrations that are generated in the body of a human or an animal.

What is claimed is:

1. A biosensor comprising:
a sheet-like piezoelectric element;
a spacer disposed around the piezoelectric element in plan view with a gap therebetween; and
a covering member that covers a front side of each of the spacer and the piezoelectric element,
the spacer supporting the covering member from a rear side of the covering member, and
the piezoelectric element being fixed to the covering member.

2. The biosensor according to claim 1, wherein
the rear side of the covering member faces toward a surface of a living body to be detected for vibration, and
the front side of each of the spacer and the piezoelectric element faces away from the surface of the living body.

3. The biosensor according to claim 1, wherein
a rear surface on a rear side of the spacer is a plane that is parallel to a rear surface on a rear side of the piezoelectric element, the rear side of the spacer is opposite to the front side of the spacer, and the rear side of the piezoelectric element is opposite to the front side of the piezoelectric element.

4. The biosensor according to claim 1, further comprising
a plate disposed on a rear side of the piezoelectric element so as to face toward the covering member, and the rear side of the piezoelectric element is opposite to the front side of the piezoelectric element.

5. The biosensor according to claim 4, wherein
a rear surface on a rear side of the plate projects rearward from a rear surface on a rear side of the spacer, the rear side of the spacer is opposite to the front side of the spacer, and the rear side of the plate faces in the same direction as the rear side of the spacer.

6. The biosensor according to claim 1, further comprising
at least one additional piezoelectric element arranged so as not to overlap the piezoelectric element in the plan view.

7. The biosensor according to claim 1, wherein
a mean thickness of the spacer is 300 μm or more and 800 μm or less.
